(19) United States
(12) Patent Application Publication
(10) Pub. No.: US 2023/0061278 A1
(43) Pub. Date: Mar. 2, 2023

(54) METHOD FOR FIXING THE GUIDE SHEATH FOR CABLES FOR ACTUATING THE DISTAL HEAD OF A MEDICAL DEVICE

(71) Applicant: AXESS VISION TECHNOLOGY, (US)

(72) Inventor: Emmanuel HALLAUER, SACHE (FR)

(21) Appl. No.: 17/793,057

(22) PCT Filed: Jan. 15, 2021
(86) PCT No.: PCT/FR2021/050074

§ 371 Date: Jul. 15, 2022

(30) Foreign Application Priority Data
Jan. 17, 2020 (FR) FR2000452

(51) Int. Cl. A61B 1/00; A61B 1/005; A61B 1/01; B29C 65/08

(52) U.S. Cl. CPC A61B 1/0011; A61B 1/0052; A61B 1/01; B29C 65/08

(57) ABSTRACT

A method for fixing a guide sheath of a cable for actuating the distal head of a medical device including providing a housing having at least one column made of a thermoplastic material for fixing a support sheath, arranging, from the terminal portion of the column, a groove that extends radially therethrough to allow the insertion of the support sheath, positioning the support sheath of the actuating cable inside the groove while a free end extends out of the column, and heating at least the free end of the column and applying a pressure to the column to ensure the attachment of the support sheath to the housing by means of welding.

### TECHNICAL FIELD

The present disclosure relates to the technical field of medical devices in the general sense allowing access to the interior of a body such as a cavity or a channel for example and it is more specifically aimed at medical devices of the catheter type and preferably, medical devices of the endoscope type.

The object of the disclosure finds a particularly advantageous application for reusable or single-use endoscopes.

The present disclosure relates more specifically to the fixing of the guide sheaths of the actuating cables of the bending structure allowing to orient the distal head of such medical devices of the catheter or endoscope type, this distal head being adapted to ensure multiple functions such as visualization, supply of fluid, aspiration of fluid, supply of instruments, taking samples or performing surgical operations for example.

The medical device of the catheter or endoscope type in accordance with the disclosure finds particularly advantageous applications for providing access to the internal surface of a hollow organ, of a cavity or of a natural or artificial conduit of the human body to perform various operations for therapeutic, surgical or diagnostic purposes.

The medical device of the catheter or endoscope type according to the disclosure is used for diagnostic, therapeutic or surgical purposes for the inspection of all the internal portions of the human body accessible by natural or artificial means. For example, the medical device of the catheter or endoscope type according to the disclosure can be used in the field of the urinary tract, the gastrointestinal tract, the respiratory system, the cardiovascular system, the trachea, the sinus cavity, the female reproductive system, abdominal cavity or any other portion of the human body to be explored by natural or artificial means.

### BACKGROUND

In general, a medical endoscope includes, as described for example by patent application WO 2014/106510, a control handle to which an insertion tube is fixed. This tube includes a distal head equipped with an optical viewing system to illuminate and examine the organ, cavity or conduit of the human body. Upstream of this distal head, the insertion tube includes a bending structure or deflection portion formed of the articulated vertebrae allowing the orientation of the distal head using one or more actuating cables mounted inside the insertion tube. Each actuating cable includes a distal end fixed to the distal head and a proximal end on which acts a control mechanism equipping the handle to ensure the sliding of the cables and consequently the folding of this deflection portion in order to orient the distal head.

Conventionally, each actuating cable is mounted inside a guide sheath fixed to the control handle so that the movement imposed on the proximal end of the cable relative to the guide sheath, using the control mechanism, can be transmitted to the distal end of the actuating cable causing a corresponding movement of the distal head. The guide sheath is fixed to the control handle, for example by gluing as described in patent application WO 2016/188537. Such a solution does not give satisfaction in practice because it appears difficult to automate such a fixing operation to obtain a constant quality of fixing over an entire production. Moreover, this type of fixing does not have all the guarantees of resistance to aging, for example during storage and transport. Other solutions include holding the cables in position in a nut inserted into a slider as described in document EP 1 737 335, or using pulleys as described in U.S. Pat. No. 6,673,012.

The use of a trigger to actuate the distal portion of an endoscope has also been described in document EP 3 066 995.

### SUMMARY

The present disclosure therefore aims at overcoming the disadvantages of the state of the art by proposing a new technique for fixing to the control handle, the guide sheaths for the cables for actuating the distal head of a medical device of the catheter or endoscope type, this new technique ensuring a long-lasting and constant quality of fixation for the whole of a production while having a low cost of implementation.

To achieve such an objective, the method for fixing a guide sheath of a cable for actuating the distal head of a medical instrument includes the following steps:
providing a housing of a control handle for a medical instrument, having at least one column made of a thermoplastic material for fixing a support sheath of an actuating cable movable in translation relative to the support sheath;
arranging from the terminal portion of the column a groove that extends radially therethrough to allow the insertion of the support sheath of the actuating cable;
positioning the support sheath of the actuating cable inside the groove, while a free end extends out of the column;
heating at least the free end of the fixing column and applying a pressure to the column to ensure the attachment of the support sheath to the housing of the control handle by means of welding.

Advantageously, pressure is exerted on the column to move the material until a bead in contact with the support sheath is obtained. Thus, the attachment of the support sheath to the housing is better.

Preferably, pressure is exerted on the column until a bead which at least partially closes the groove is obtained.

Advantageously, the housing of the control handle and the column are made of a thermoplastic material.

According to an exemplary embodiment, the groove is arranged from the terminal portion of the column with a abutment bottom for inserting the support sheath into the groove, the depth of the groove being such that the free end of the column extends beyond the support sheath.

The groove is arranged with a depth equal to a value included between 1.5 to 5 times the diameter of the support sheath.

According to an advantageous variant embodiment, ultrasonic welding ensures the attachment of the support sheath to the housing of the control handle.

The method according to the disclosure implements hot and pressure riveting to ensure the attachment of the support sheath to the housing of the control handle.

Another object of the disclosure is to provide a housing of a control handle for a medical device of the catheter or endoscope type, provided with at least one column made of thermoplastic material and provided with a radial through groove in which is mounted a support sheath of an actuating cable movable in translation relative to the housing, the column having a deformed free end in the shape of bead made of a thermoplastic material and cooperating with the support sheath to connect the support sheath to the housing.

Advantageously, the column and the housing are made of a thermoplastic material.

Various other features emerge from the description given below with reference to the appended drawings which show, by way of non-limiting examples, embodiments of the object of the disclosure.

### BRIEF DESCRIPTION OF THE DRAWINGS

FIG. 1 is a schematic view of a medical device of the catheter or endoscope type in the general sense including a control handle provided with fixing columns for the guide sheath of the cables for actuating the distal head of the medical device.

FIG. 2 is a perspective view showing the positioning of the guide sheath of an actuating cable, before fixing it to the control handle.

FIG. 2A is a cross-sectional view taken along lines A-A of FIG. 2.

FIG. 3 is a perspective view showing the guide sheath of the actuating cable after it has been fixed to the control handle.

FIG. 3A is a cross-sectional view taken along lines A-A of FIG. 3 showing the guide sheath of the actuating cable fixed to the control handle.

### DESCRIPTION OF EMBODIMENTS

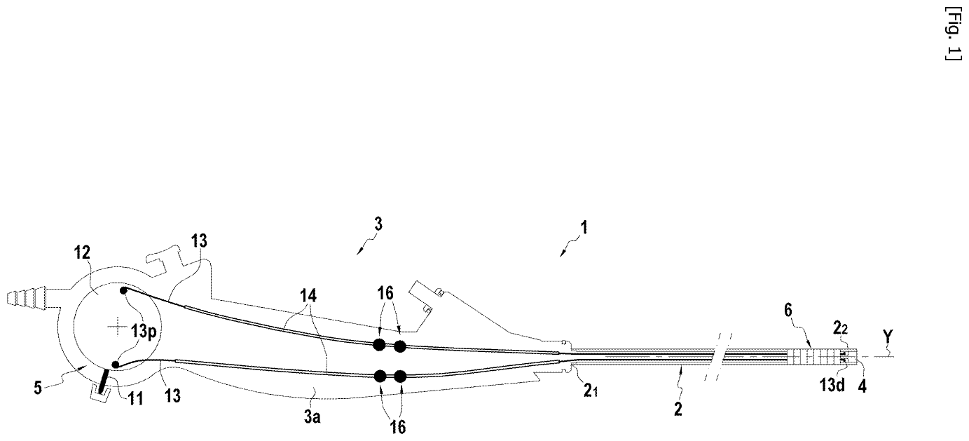

FIG. 1 illustrates by way of example, a medical device 1 of the endoscope or catheter type in the general sense designed to access the interior of a body such as a cavity or a channel for example. Conventionally, a medical device 1 of the endoscope or catheter type includes an insertion tube 2 having, on one side, a proximal portion 2₁ connected to the housing 3a of a control handle 3 and, on the opposite side, a distal portion 2₂, which is equipped with a distal head 4. The insertion tube 2 is fixed temporarily or permanently on the housing of the control handle 3. This insertion tube 2 which has a length and a flexibility which is more or less significant is intended to be introduced into a natural or artificial access route in order to perform various operations or functions for therapeutic, surgical or diagnostic purposes. The insertion tube 2 is made of a semi-rigid material and has a length adapted to the length of the conduit to be inspected and which can be included between 5 cm and 2 m. The insertion tube 2 has various cross-sectional shapes such as square, oval or circular. This insertion tube 2 which is in contact with tissues, human organs or medical equipment (trocars or probes), is essentially of single use or multiple use of a patient or even a reusable use after decontamination, disinfection or sterilization.

According to a preferred embodiment, the medical device 1 according to the disclosure is an endoscope including a vision system able to illuminate and bring back an image of the distal portion of the insertion tube 2. The endoscope 1 thus includes a vision system mounted inside the control handle 3 and penetrating inside the insertion tube 2 as far as the distal head 4.

Conventionally, the medical device 1 also includes a control mechanism 5 allowing to orient the distal head 4 with respect to the longitudinal axis Y of the insertion tube 2. To this end, the insertion tube 2 includes upstream of the distal head 4, a bending, folding or deflection portion 6 allowing the orientation of the distal head 4 with respect to the longitudinal axis Y of the insertion tube 2. This bending, folding or deflection portion 6 can be made in any appropriate way to ensure the flexion of the distal head 4 with respect to the longitudinal axis Y of the insertion tube 2. For example, this bending, folding or deflection portion 6 can be produced by a spring or by tubular vertebrae articulated together.

The control mechanism 5 can be made in any suitable way so that the distal head 4 can be moved between a rest position in which the insertion tube 2 is straight and a bent position in which the deflection portion 6 is curved. By way of non-limiting example, the control mechanism 5 may correspond to the control mechanism described in patent FR 3 047 887. To this end, the control mechanism 5 includes a manual control lever 11 accessible from the outside of the housing 3a of the control handle. This control lever 11 causes the rotation at least one pivoting part such as a pulley 12 mounted inside the housing 3a and on which is fixed at least one and in the example illustrated, two actuating cables 13 mounted inside the insertion tube 2 to be fixed to the distal head 4. The proximal ends 13p of the actuating cables 13 are fixed symmetrically opposite on the pulley 12 with respect to a diametrical plane while the distal ends 13d of the actuating cables 13 are fixed to the distal head 4.

This control mechanism 5 is adapted to ensure, for example, the left-right or up-down movement of the distal head 4. In the example illustrated, the control lever 11 has a rotational stroke but it is possible that the control lever 11 has a translational movement causing the rotation of the pivoting part 12 via a movement transformation system. Similarly, the control mechanism 5 can be adapted to ensure the left-right and up-down movement of the distal head 4 using three or four actuating cables 13.

Of course, the actuating cables 13 are made in any suitable way to ensure the movement transmission function leading to the flexion of the distal head 4. Thus, these actuating cables 13 can be made for example by rods, threads, filaments, strands or chains, made of a metal or polymer material for example. Typically, the actuating cables 13 are made of stainless steel with a diameter for example included between 0.1 mm and 2 mm.

Each actuating cable 13 is mounted inside a support sheath 14 intended to be fixed to the housing 3a of the control handle 3. The support sheath 14 thus ensures the sliding guidance of the actuation cable 13 during the operations of deflection of the distal head 4. The actuating cable 13 thus moves in translation with respect to the support sheath 14 which is mounted secured with the control handle 3. For example the support sheath 14 is made of stainless steel. Typically, the support sheath 14 is formed by winding one or more wires along the actuating cable 13.

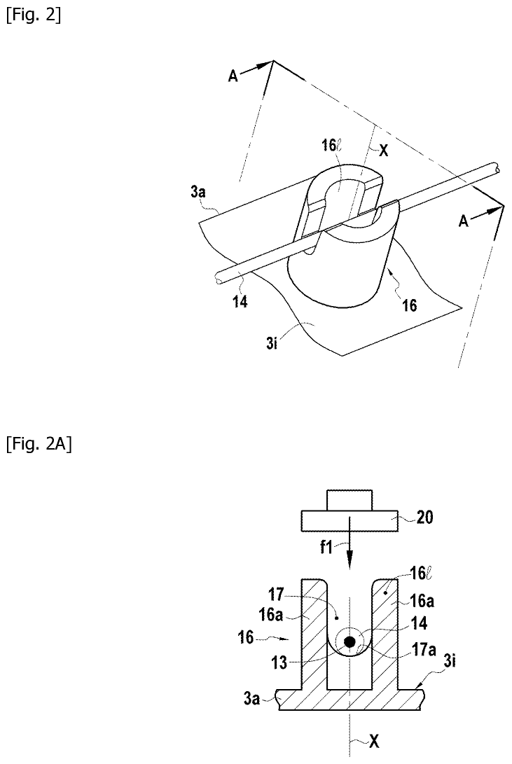

According to a feature of the disclosure, the housing 3a of the control handle 3 is provided internally with at least one protrusion or protuberance 16 projecting from the internal face 3i of the housing 3a of the control handle 3 (FIGS. 2, 2A). This protrusion or protuberance 16 is designated in the following description by column 16 thus corresponding to a chimney, a stud or a pad rising from the internal face 3i of the housing 3a in a direction of extension X preferably substantially perpendicular to the inner face 3i of the housing 3a. As will be described in detail in the following description, each column 16 ensures the attachment of the support sheath 14 to the housing 3a of the control handle 3 while allowing the sliding of the actuating cable 13 relative to the support sheath 14. Each column thus provides a fixing of the support sheath 14 with the housing 3a which is solid and permanent.

In the example illustrated in FIG. 1, the control mechanism 5 includes two actuating cables 13 each mounted inside a support sheath 14 fixed to the housing 3a using two fixing columns 16 in accordance with the disclosure. Of course, each support sheath 14 can be anchored to the housing 3a using a different number of fixing columns 16.

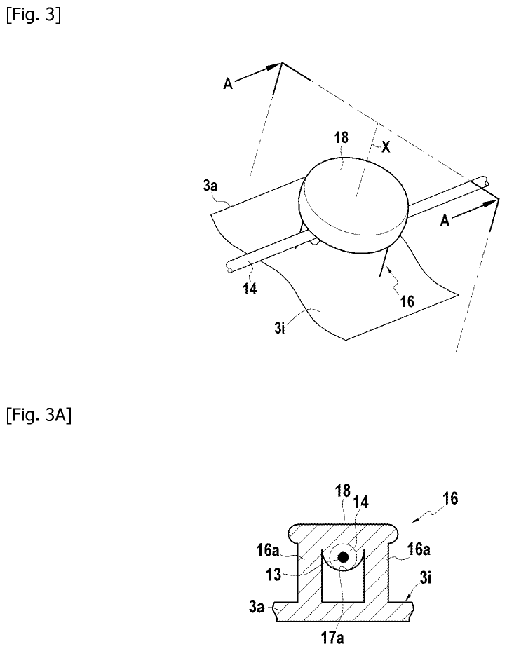

Each column 16 is crossed from side to side by a radial groove 17 which, before fixing the support sheath 14 to the column, is open at the terminal portion of the column 16 to allow engagement, from the terminal portion of the column 16, of the support sheath 14 of an actuating cable 13. The groove 17 is arranged from the terminal portion of the column 16 extending in a secant direction to the direction of extension X, for example substantially perpendicular to the direction of extension X, opening onto two opposite sides of the column 16. As emerges more specifically from FIG. 3A, the groove 17 includes a bottom 17a on which rests the support sheath 14. In the example illustrated in the drawings, each column 16 is made in the shape of a tube crossed radially by a groove 17. It is clear that the column 16 can be made by a solid cylinder crossed radially by a groove 17.

After fixing the support sheath 14 to the column, each column 16 has two branches 16a rising on either side of the groove 17 to be connected, opposite the housing 3a, by a bead 18 of material identical to the material of the column 16. This bead 18 of material at least partially fills the groove 17 to cooperate with the support sheath 14 and thus connect the support sheath 14 to the housing 3a. In other words, this bead 18 is in contact with the support sheath 14 while fitting into the thickness of the support sheath 14 from its external surface. The bead 18 thus penetrates superficially into the thickness of the support sheath 14. In the case where the support sheath 14 is formed by a winding of one or more wires, the bead 18 is inserted between the windings of the wire(s) of the support sheath 14. The support sheath 14 is thus securely anchored to the housing 3a.

According to one feature of the disclosure, the support sheath 14 is attached by welding to the column 16. According to one feature of the disclosure, the columns 16 are made of a thermoplastic material, that is to say a material capable of being softened by heating above a certain temperature and hardened by cooling. For example, the material in which the columns 16 are made falls under the category of semi-crystalline or amorphous thermoplastic polymers. Typically, the columns 16 are for example made of ABS (acrylonitrile butadiene styrene), PP (polyprolylene), POM (polyoxymethylene), polyamide, polyurethane, PE (polyethylene), PS (polystyrene), or PVC (poly(vinyl chloride)), or any mixture of these polymers.

According to an advantageous embodiment, the housing 3a is made of a thermoplastic material. According to a preferred variant embodiment, the housing 3a and the columns 16 are made from the same thermoplastic material. Typically, the housing 3a is manufactured using the molding technique during which the columns 16 and the housing 3a are formed simultaneously. Of course, it could be considered to add the columns 16 onto the internal face 3i of the housing 3a.

The fixing of a guide sheath 14 to the housing 3a using the fixing columns 16 is carried out by the method described below.

The first step of the fixing method includes providing a housing 3a of a control handle for a medical device 1 of the endoscope or catheter type, having at least one column 16 of thermoplastic material, adapted to allow fixing a support sheath 14 of an actuating cable 13 movable in translation relative to the support sheath. In the example illustrated, the housing 3a includes for each of the two support sheaths 14, two columns 16 rising close to each other, from the internal face 3i of the housing 3a and each made in the shape of a shaft or a tubular chimney. Of course, the number of fixing columns 16 for each of the support sheaths 14 can be different from two and the number of support sheaths 14 equipping such a control handle can be different from two.

Advantageously, the housing 3a and the columns 16 are made in the same manufacturing step. The housing 3a and the columns 16 are thus produced in a thermoplastic material and preferably made of the same thermoplastic material. However, a step aiming at adding the columns 16 onto the housing 3a by fixing them by any appropriate means to the housing 3a cannot be excluded.

From the terminal portion of the column 16, a radially traversing groove 17 is arranged to allow the insertion of the support sheath 14 of the actuating cable 13. This groove 17 is open in the opposite direction from the internal face 3i of the housing 3a to allow the engagement of the support sheath 4 from the terminal portion of the column 16. This groove 17 is preferably made during the manufacturing step of the column 16. Advantageously, the housing 3a and the columns 16 provided with the grooves 17 are made in the same manufacturing step. Typically, the housing 3a and the columns 16 provided with the grooves 17 are made by injection molding. Of course, this groove 17 can be made during a step subsequent to the actual manufacturing step of the column 16.

According to the embodiment illustrated in the drawings, the groove 17 has a bottom 17a rising above the inner face 3i of the housing 3a. Of course, the bottom 17a of the groove 17 can correspond to the internal face 3i of the housing 3a. The bottom 17a of the groove 17 is capable of forming an abutment or bearing surface for the support sheath 14.

According to one embodiment feature, the groove 17 is made with a determined depth so that once the support sheath 14 is inserted into the groove, the column 16 has a free end 16l extending beyond or above the support sheath 14. This free end 16l corresponds to the portion of the branches 16a which extends beyond or above the support sheath 14. In other words, the depth of the groove 17 is strictly greater than the diameter of the support sheath 14. For example, the groove 17 is arranged with a depth equal to a value included between 1.5 and 5 times the diameter of the support sheath 14. Each column 16 thus includes two branches 16a rising on either side of the groove 17, each having a free end 16l projecting from the support sheath 14.

The fixing method thus includes positioning the support sheath 14 inside the groove 17 set back from the terminal portion of the column 16, that is to say while the free end 16I of the column extends out of the support sheath 14. The step of inserting the support sheath 14 inside the groove 17 can be carried out manually or automatically using a robotic arm. According to an advantageous feature, the insertion of the support sheath 14 inside the groove 17 is carried out until the support sheath abuts on the bottom 17a of the groove 17.

The fixing method includes heating at least the free end of the fixing column 16 and in applying a pressure on the column 16 to ensure, by welding, the fixing of the support sheath 14 to the housing 3a of the control handle 3. As shown in FIG. 2A, at least the pressure force is exerted using a welding head 20 adapted to the welding method implemented. Indeed, the heat can be supplied by the welding head 20 and/or by a device independent of the welding head 20. In the same way, the heat can be supplied before the application of the pressure force or simultaneously to the application of the pressure force. This step of welding the support sheath 14 inside the groove 17 can be carried out manually or automatically using a robotic arm. For example, the displacement parameters of the welding head 20 as well as the temperature parameters which can be defined to obtain effective welding can be reproduced for all the columns 16 of the housings 3a.

The supply of heat at the free end 16l of the column 16 leads to its softening so that the material is moved under the effect of the application of a pressure according to a direction of coming closer to the welding head 20 in the direction of the support sheath 14, represented by the arrow fl in FIG. 2A. The direction of movement of the welding head 20 is advantageously established parallel to the direction of extension X of the column 16. In the example illustrated, the welding head 20 bears on the terminal portion of the free end 16l of the column 16. Advantageously, the welding head 20 is moved relative to the column 16. Of course, it can be considered to move the column 16, namely the housing 3a relative to the welding head 20.

The displaced thermoplastic material contacts or cooperates with the support sheath 14 which thus becomes secure with the column 16. At least part of the displaced thermoplastic material thus penetrates inside the groove 17 (FIG. 3A). According to an advantageous variant of the fixing method, pressure is exerted on the fixing column 16 so as to move the softened material until obtaining a protrusion, a bead or a stud 18 in contact with the support sheath 14 or penetrating the support sheath 14 on the surface. In the case where the support sheath 14 is formed by a winding of one or more wires, the softened material of this protrusion is inserted between the windings of the wire(s) of the support sheath 14.

This bead 18 comes from the thermoplastic material resulting from the deformation of at least the free end 16l of the column 16. This bead 18 of course remains attached to the column 16 forming a single piece. Compared to its state before deformation, the column 16 is deformed according to a greater or lesser height of its free end 16l.

According to an advantageous variant of the fixing method, pressure is exerted on the column 16 until a bead 18, at least locally closing the groove 17 is obtained. In the drawings, the displaced thermoplastic material thus forms a solid disc above the support sheath 14 completely closing the groove 17. Of course, the bead 18 can take a different shape such as the shape of a ring for example being established in correspondence with the tubular shape of the column 16.

The fixing of the support sheath 14 to the housing 3a is carried out by the implementation of a hot riveting method, namely (laser, induction, hot air, infra-red for example) or a (orbital, longitudinal or axial) friction vibration welding method.

According to an exemplary implementation, the attachment of the support sheath 14 to the housing 3a is carried out by the implementation of an ultrasonic welding method. According to this embodiment, the housing 3a is for example positioned on an anvil and the welding head 20 such that a vibrating sonotrode is applied to the terminal portion of the column 16 by being moved a few mm to move the material.

The method according to the disclosure allows to obtain a housing 3a of a control handle for a medical device 1 of the endoscope or catheter type including at least one column 16 ensuring the fixing of a support sheath 14 for a cable for actuating the distal head 4 of this medical device 1. This fixing is solid and permanent.

The disclosure is not limited to the examples described and represented because various modifications can be made thereto without departing from its scope.

1. A method for fixing a guide sheath of a cable configured for actuating a distal head of a medical device, the method comprising:
providing a housing of a control handle configured for a medical device, having at least one column made of a thermoplastic material for fixing a support sheath of an actuating cable movable in translation relative to the support sheath;
arranging, from a terminal portion of the column, a groove that extends radially therethrough to allow the insertion of the support sheath of the actuating cable;
positioning the support sheath of the actuating cable inside the groove, while a free end extends out of the column; and
heating at least the free end of the fixing column and applying a pressure to the column to ensure the attachment of the support sheath to the housing of the control handle by means of welding.

2. The fixing method according to claim 1, according to which pressure is exerted on the column to move the material until a bead in contact with the support sheath is obtained.

3. The fixing method according to claim 1, according to which pressure is exerted on the column until a bead which at least partially closes the groove is obtained.

4. The fixing method according to claim 1, wherein the housing of the control handle and the column are made of a thermoplastic material.

5. The fixing method according to claim 1, according to which the groove is arranged from the terminal portion of the column with an abutment bottom for inserting the support sheath into the groove, the depth of the groove being such that the free end of the column extends beyond the support sheath.

6. The fixing method according to claim 5, according to which the groove is arranged with a depth equal to a value comprised between 1.5 and 5 times a diameter of the support sheath.

7. The fixing method according claim 1, wherein ultrasonic welding ensures the attachment of the support sheath to the housing of the control handle.

8. The fixing method according to claim 1, according to which heat and pressure riveting ensures the attachment of the support sheath to the housing of the control handle.

9. A housing configured for a control handle of a medical instrument provided with at least one column made of thermoplastic material and provided with a radial through groove in which is mounted a support sheath of an actuating cable movable in translation relative to the housing, the column having a deformed free end in the shape of a bead made of thermoplastic material and cooperating with the support sheath to connect the support sheath to the housing.

10. The housing according to claim 9, wherein the column and the housing are made of a thermoplastic material.
